(19) United States
(12) Patent Application Publication
(10) Pub. No.: US 2017/0224670 A1
(43) Pub. Date: Aug. 10, 2017

(54) Drug Combination to Treat Melanoma

(71) Applicant: Exelixis, Inc., South San Francisco, CA (US)

(72) Inventor: Keiran Smalley, Tampa, FL (US)

(73) Assignee: Exelixis, Inc., South San Francisco, CA (US)

(21) Appl. No.: 15/519,012

(22) PCT Filed: Oct. 14, 2015
(86) PCT No.: PCT/US15/55568

§ 371 Date: Apr. 13, 2017

Related U.S. Application Data
(60) Provisional application No. 62/063,755, filed on Oct. 14, 2014.

(51) Int. Cl. A61K 31/46; A61K 31/437

(52) U.S. Cl. CPC A61K 31/46; A61K 31/437

(57) ABSTRACT

This invention relates to the combination of vemurafenib and the heat shock protein 90 (HSP90) inhibitor XL888 to treat melanoma, particularly BRAF V600 mutant melanoma in patients in need of such treatment.

### PRIORITY CLAIM

This application claims priority to U.S. Provisional Application Ser. No. 62/063,755, filed Oct. 14, 2014. The entire contents of the aforementioned application are incorporated herein by reference.

### FIELD OF INVENTION

This invention relates to the combination of vemurafenib and the heat shock protein 90 (HSP90) inhibitor XL888 to treat melanoma, particularly metastatic BRAF V600 mutant melanoma in patients in need of such treatment.

### BACKGROUND OF THE INVENTION

According to the American Cancer Society, cancer of the skin is by far the most common type of cancer. Melanoma accounts for less than 2 percent of skin cancer cases but causes a majority of skin cancer deaths. In the United States approximately 76,100 new melanomas will be diagnosed in 2014, and approximately 9,710 people are expected to die of melanoma. While patients with early-stage melanomas can be treated successfully with surgery, a substantial minority will develop stage IV melanoma. Historically, the prognosis for patients with stage IV melanoma has been poor. The median survival is less than 1 year and the 5-year overall survival rate is less than 10 percent.

Prior to 2011, Dacarbazine and interleukin-2 (IL-2) were used in the United States for the treatment of patients with stage IV melanoma. More recently, the discovery that approximately half of all melanomas have mutations in the BRAF gene has spurred the development of BRAF inhibitors to treat melanoma. Most notable of these is vemurafenib (Zelboraf).

Vemurafenib has been approved by the FDA at 960 mg dosed twice daily for the treatment of patients with unresectable or metastatic melanoma with BRAF V600 mutation. However, in spite of the gains in survival made by vemurafenib in most BRAF mutation-associated melanomas, researchers have observed vemurafenib resistance in many patients taking the drug. On average, resistance develops within 7 months of initial use. Resistance is generally not because of further adaptive mutations in B-RAF but rather mutations in genes coding for other proteins.

As a result, there is a need to develop new therapies that limit or prevent chemotherapy resistance in patients with melanoma who are being treated with vemurafenib.

### SUMMARY OF THE INVENTION

These and other needs are met by the present invention which is directed to a method of treating unresectable or metastatic melanoma with BRAF V600 mutation (“BRAF V600 mutant melanoma”) comprising administering to a patient in need of such treatment a combination comprising an effective amount of vemurafenib in combination with an effective amount of the heat shock protein90 (HSP90) inhibitor XL888 or a pharmaceutically acceptable salt thereof.

In one aspect, the invention relates to method of treating BRAF V600 mutant melanoma comprising administering to a patient in need of such treatment a combination comprising vemurafenib in combination with the heat shock protein90 (HSP90) inhibitor XL888 or a pharmaceutically acceptable salt thereof, wherein vemurafenib is dosed orally at up to 960 mg twice daily and XL888 is dosed orally at up to 120 mg twice a week. In one embodiment of this aspect, XL888 is dosed orally at up to 105 mg twice a week. In another embodiment, XL888 is dosed orally at up to 90 mg twice a week. In another embodiment of this aspect, XL888 is dosed orally at up to 75 mg twice a week. In another embodiment of this aspect, XL888 is dosed orally at up to 60 mg twice a week. In another embodiment of this aspect, XL888 is dosed orally at up to 45 mg twice a week. In another embodiment of this aspect, XL888 is dosed orally at up to 30 mg twice a week.

In one embodiment, the vemurafenib is administered as a twice daily dose of up to 960 mg and the heat shock protein (HSP90) inhibitor XL888 or a pharmaceutically acceptable salt thereof is administered as a twice weekly dose of 90 mg or less.

The structure of vemurafenib is shown below.

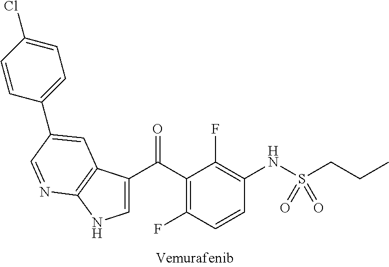 [SMILES: CCCS(=O)(=O)Nc1ccc(F)c(C(=O)c2c[nH]c3ncc(-c4ccc(Cl)cc4)cc23)c1F]

Dosing and treatment-related information regarding vemurafenib is available from the FDA, at www.accessdata.fda.gov/scripts/cder/drugsatfda/index.cfm?fuseaction=Search.DrugDetails (last visited Oct. 14, 2014).

The structure of XL888 is shown below.

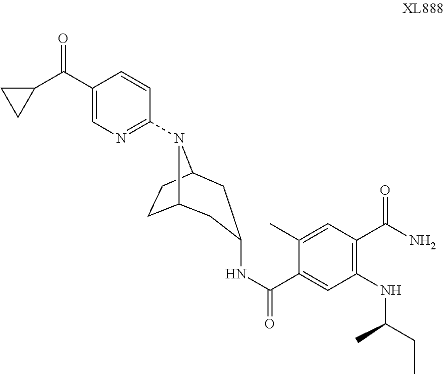

XL888 is specifically disclosed and claimed in WO 2009/055077, the entire contents of which is incorporated herein by reference. XL888 is additionally described in Bioorganic & Medicinal Chemistry Letters, 22 (2012) 5396-5404.

XL888 is a potent small molecule inhibitor of HSP90. HSP90 client proteins include wildtype or mutant oncoproteins required for tumor cell growth and survival, including HER2, EGFR, CDK4, CRAF, AKT, c-MET, BRAF, HIF-1α, and p53. HSP90 clients are molecular intermediates that mediate resistance to BRAF inhibitors such as vemurafenib. HSP90 inhibition may abrogate both the early therapeutic escape and the complexity of acquired BRAFi resistance. To that end, the signaling proteins implicated in vemurafenib resistance are clients of HSP90. Inhibition of HSP90 by XL888 can help to restore sensitivity to vemurafenib. Clinical Cancer Research, 2012; DOI: 10.1158/1078-0432.CCR-11-2612.

The combination of vemurafenib with XL888 according to the dosages and schedules disclosed herein possess one or more desirable attributes, including improvements in the anticancer profile of the combination as compared to single agent treatment; similar or reduced side effect profile of the combination as compared to single agent treatment; similar or reduced dosing load for the combination as compared to single agent treatment; and reduced acquired resistance for the combination as compared to single agent treatment.

### DETAILED DESCRIPTION

As indicated above, this disclosure relates to a method of treating BRAF V600 mutant melanoma comprising administering an effective amount of vemurafenib in combination with an effective amount of the HSP90 inhibitor XL888 or a pharmaceutically acceptable salt thereof.

As used herein, the term “pharmaceutically acceptable salt” refers to a salt prepared from XL888 and a pharmaceutically acceptable inorganic or organic acid. Suitable acids include for example hydrochloric acid, hydrobromic acid, sulfuric acid, nitric acid, phosphoric acid, and the like, as well as organic acids such as acetic acid, trifluoroacetic acid, propionic acid, glycolic acid, pyruvic acid, oxalic acid, malic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid and the like.

XL888 is typically administered to patients as a capsule or tablet comprising a pharmaceutically acceptable carrier. A pharmaceutically acceptable carrier may contain inert ingredients which do not unduly inhibit the biological activity of the compound(s) described herein. The pharmaceutically acceptable carriers should be biocompatible, i.e., non-toxic, non-inflammatory, non-immunogenic and devoid of other undesired reactions upon the administration to a subject. Standard pharmaceutical formulation techniques can be employed, such as those described in REMINGTON, J. P., REMINGTON'S PHARMACEUTICAL SCIENCES (Mack Pub. Co., 17^{th} ed., 1985). Suitable pharmaceutical carriers for parenteral administration include, for example, sterile water, physiological saline, bacteriostatic saline (saline containing about 0.9% mg/ml benzyl alcohol), phosphate-buffered saline, Hank's solution, Ringer's-lactate, and the like. Methods for encapsulating compositions, such as in a coating of hard gelatin or cyclodextran, are known in the art. See BAKER, ET AL., CONTROLLED RELEASE OF BIOLOGICAL ACTIVE AGENTS, (John Wiley and Sons, 1986).

As used herein, the term “effective amount” refers to an amount of a compound described herein which is sufficient to reduce or ameliorate the severity, duration, progression, or onset of a disease or disorder, delay onset of a disease or disorder, retard or halt the advancement of a disease or disorder, cause the regression of a disease or disorder, prevent or delay the recurrence, development, onset or progression of a symptom associated with a disease or disorder, or enhance or improve the therapeutic effect(s) of another therapy. In one embodiment of the invention, the disease or disorder is a proliferative disorder. The precise amount of compound administered to a subject will depend on the mode of administration, the type and severity of the disease or condition and on the characteristics of the subject, such as general health, age, sex, body weight and tolerance to drugs. For example, for a proliferative disease or disorder, determination of an effective amount will also depend on the degree, severity and type of cell proliferation. The skilled artisan will be able to determine appropriate dosages depending on these and other factors. When co-administered with other therapeutic agents, e.g., when co-administered with an anti-cancer agent, an “effective amount” of any additional therapeutic agent(s) will depend on the type of drug used. Suitable dosages are known for approved therapeutic agents and can be adjusted by the skilled artisan according to the condition of the subject, the type of condition(s) being treated and the amount of a compound being used. In cases where no amount is expressly noted, an effective amount should be assumed. Non-limiting examples of an effective amount of a compound described herein are provided herein below.

As used herein, the terms “treat”, “treatment” and “treating” refer to the reduction or amelioration of the progression, severity and/or duration of a disease or disorder, delay of the onset of a disease or disorder, or the amelioration of one or more symptoms (preferably, one or more discernible symptoms) of a disease or disorder, resulting from the administration of one or more therapies (e.g., one or more therapeutic agents such as a compound of the invention). The terms “treat”, “treatment” and “treating” also encompass the reduction of the risk of developing a disease or disorder, and the delay or inhibition of the recurrence of a disease or disorder. In one embodiment, the disease or disorder being treated is a proliferative disorder such as cancer. In specific embodiments, the terms “treat”, “treatment” and “treating” refer to the amelioration of at least one measurable physical parameter of a disease or disorder, such as growth of a tumor, not necessarily discernible by the patient. In other embodiments the terms “treat”, “treatment” and “treating” refer to the inhibition of the progression of a disease or disorder, e.g., a proliferative disorder, either physically by the stabilization of a discernible symptom, physiologically by the stabilization of a physical parameter, or both. In another embodiment, the terms “treat”, “treatment” and “treating” of a proliferative disease or disorder refers to the reduction or stabilization of tumor size or cancerous cell count, and/or delay of tumor formation. In another embodiment, the terms “treat”, “treating” and “treatment” also encompass the administration of a compound described herein as a prophylactic measure to patients with a predisposition (genetic or environmental) to any disease or disorder described herein.

As used herein, the term “synergistic” refers to a combination of a compound described herein and another therapeutic agent, which, when taken together, is more effective than the additive effects of the individual therapies. A synergistic effect of a combination of therapies (e.g., a combination of therapeutic agents) permits the use of lower dosages of one or more of the therapeutic agent(s) and/or less frequent administration of the agent(s) to a subject with a disease or disorder, e.g., a proliferative disorder. The ability to utilize lower the dosage of one or more therapeutic agent and/or to administer the therapeutic agent less frequently reduces the toxicity associated with the administration of the agent to a subject without reducing the efficacy of the therapy in the treatment of a disease or disorder. In addition, a synergistic effect can result in improved efficacy of agents in the prevention, management or treatment of a disease or disorder, e.g. a proliferative disorder. Finally, a synergistic effect of a combination of therapies may avoid or reduce adverse or unwanted side effects associated with the use of either therapeutic agent alone.

As used herein, the phrase “side effects” encompasses unwanted and adverse effects of a therapeutic agent. Side effects are always unwanted, but unwanted effects are not necessarily adverse. An adverse effect from a therapeutic agent might be harmful or uncomfortable or risky to a subject. Side effects include fever, chills, lethargy, gastrointestinal toxicities (including gastric and intestinal ulcerations and erosions), nausea, vomiting, neurotoxicities, nephrotoxicities, renal toxicities (including such conditions as papillary necrosis and chronic interstitial nephritis), hepatic toxicities (including elevated serum liver enzyme levels), myelotoxicities (including leukopenia, myelosuppression, thrombocytopenia and anemia), dry mouth, metallic taste, prolongation of gestation, weakness, somnolence, pain (including muscle pain, bone pain and headache), hair loss, asthenia, dizziness, extra-pyramidal symptoms, akathisia, cardiovascular disturbances, visual disturbance, and sexual dysfunction.

As used herein, the term “in combination” refers to the use of more than one therapeutic agent. The use of the term “in combination” does not restrict the order in which the therapeutic agents vemurafenib and XL888 are administered to a subject with a disease or disorder, e.g., a proliferative disorder.

The first therapeutic agent, vemurafenib, is administered orally. The FDA recommended dose of vemurafenib is 960 mg taken twice daily, typically taken approximately 12 hours apart with or without a meal. Dose reductions may be necessary to manage symptomatic adverse drug reactions. Typically, dose reductions do not result in a dose below 480 mg twice daily. Thus, in one embodiment, 960 mg vemurafenib is administered twice daily to a patient. In another embodiment, 720 mg vemurafenib is administered twice daily to a patient. In another embodiment, 480 mg vemurafenib is administered twice daily to a patient.

The second therapeutic agent, XL888 or a pharmaceutically acceptable salt thereof is also administered orally. XL888 is administered twice a week. “Twice a week” means two times in every seven day period. Thus, for example, “twice a week” can mean dose 1 occurs on Day 1 and Dose 2 occurs on Day 3 of a seven-day week. Alternatively, “twice a week” can mean dose 1 occurs on Day 1 and Dose 2 occurs on Day 4 of a seven-day week. In one embodiment, the dose of XL888 or a pharmaceutically acceptable salt thereof is 30 mg twice a week. In another embodiment, the dose of XL888 or a pharmaceutically acceptable salt thereof is 45 mg twice a week. In another embodiment, the dose of XL888 or a pharmaceutically acceptable salt thereof is 60 mg twice a week. In another embodiment, the dose of XL888 or a pharmaceutically acceptable salt thereof is 75 mg twice a week. In another embodiment, the dose of XL888 or a pharmaceutically acceptable salt thereof is 90 mg twice a week. In another embodiment, the dose of XL888 or a pharmaceutically acceptable salt thereof is 105 mg twice a week. In another embodiment, the dose of XL888 or a pharmaceutically acceptable salt thereof is 120 mg twice a week.

XL888 or a pharmaceutically acceptable salt thereof may be administered prior to, concomitantly with, or subsequent to vemurafenib administration. Thus, XL888 or a pharmaceutically acceptable salt thereof can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, the administration of concomitantly with, or subsequent to vemurafenib administration.

As used herein, the terms “therapies” and “therapy” can refer to any protocol(s), method(s), and/or agent(s) that can be used in the prevention, treatment, management, or amelioration of a disease or disorder, e.g., a proliferative disorder, or one or more symptoms thereof.

As used herein, a “protocol” includes dosing schedules and dosing regimens. The protocols herein are methods of use and include therapeutic protocols.

As used herein, a composition that “substantially” comprises a compound means that the composition contains more than about 80% by weight, more preferably more than about 90% by weight, even more preferably more than about 95% by weight, and most preferably more than about 97% by weight of the compound.

The compounds described herein are defined by their chemical structures and/or chemical names. Where a compound is referred to by both a chemical structure and a chemical name, and the chemical structure and the chemical name conflict, the chemical structure is determinative of the compound's identity.

When administered to a patient (e.g., a non-human animal for veterinary use or for improvement of livestock or to a human for clinical use), the compounds described herein are administered in an isolated form, or as the isolated form in a pharmaceutical composition. As used herein, “isolated” means that the compounds described herein are separated from other components of either: (a) a natural source, such as a plant or cell, preferably bacterial culture, or (b) a synthetic organic chemical reaction mixture. Preferably, the compounds described herein are purified via conventional techniques. As used herein, “purified” means that when isolated, the isolate contains at least 95%, preferably at least 98%, of a compound described herein by weight of the isolate either as a mixture of stereoisomers, or as a diastereomeric or enantiomeric pure isolate.

Only those choices and combinations of substituents that result in a stable structure are contemplated. Such choices and combinations will be apparent to those of ordinary skill in the art and may be determined without undue experimentation.

In an additional aspect, the combination therapy described herein may comprise one or more additional anticancer therapeutic which may have the same or a different mechanism of action. For example, the combination of vemurafenib and XL888 can be modified to incorporate other treatments for melanoma. For example the combination of vemurafenib and XL888 can be modified to incorporate a MEK inhibitor. Such MEK inhibitors are known in the art and include, for example, the compound cobimetinib. Cobimetinib is disclosed in WO 2007/044515. Cobimetinib is currently in Phase III development in combination with vemurafenib to treat melanoma. In the combination, vemurafenib is dosed at 960 mg twice daily and cobimetinib is dosed at 60 mg 21 days on and 7 days off (21/7).

### EMBODIMENTS

### Embodiment 1

A method of treating u BRAF V600 mutant melanoma comprising administering to a patient in need of such treatment a combination comprising an vemurafenib in combination with the heat shock protein90 (HSP90) inhibitor XL888 or a pharmaceutically acceptable salt thereof, wherein vemurafenib is dosed orally at up to 960 mg twice daily and XL888 is dosed orally at up 90 mg twice a week.

### Embodiment 2

The method of Embodiment 1, wherein vemurafenib is dosed orally at 480 mg twice daily.

### Embodiment 3

The method of Embodiment 1, wherein vemurafenib is dosed orally at 720 mg twice daily.

### Embodiment 4

The method of Embodiments 1-3, wherein XL888 is dosed orally at 120 mg twice weekly.

### Embodiment 5

The method of Embodiments 1-3, wherein XL888 is dosed orally at 90 mg twice weekly.

### Embodiment 6

The method of Embodiments 1-3, wherein XL888 is dosed orally at 105 mg twice weekly.

### Embodiment 7

The method of Embodiments 1-3, wherein XL888 is dosed orally at 75 mg twice weekly.

### Embodiment 8

The method of Embodiments 1-3, wherein XL888 is dosed orally at 60 mg twice weekly.

### Embodiment 9

The method of Embodiments 1-3, wherein XL888 is dosed orally at 45 mg twice weekly.

### Embodiment 10

The method of Embodiments 1-3, wherein XL888 is dosed orally at 30 mg twice weekly.

### Embodiment 11

The method of Embodiments 1-8, wherein vemurafenib and XL888 are each present in a separate pharmaceutically acceptable composition and the compositions are administered simultaneously.

### Embodiment 12

The method of Embodiments 1-11, wherein vemurafenib and XL888 are each present in a separate pharmaceutically acceptable composition and the compositions are administered sequentially.

### Embodiment 13

A method of treating melanoma in a human patient comprising administering an effective amount of a combination of vemurafenib and XL888 or a pharmaceutically acceptable salt thereof to the patient, wherein the combination of vemurafenib and XL888 has greater anticancer activity against said melanoma in the human patient than the maximum tolerated dose of vemurafenib against said melanoma in the human patient or the maximum tolerated dose of XL888 alone against said melanoma in the human patient.

### Embodiment 14

The method of Embodiment 1, wherein the combination of vemurafenib and XL888 has greater anticancer activity against said melanoma in the human patient than the optimum dose of each drug alone against said melanoma in the human patient.

The invention can be understood more fully by reference to the following detailed illustrative examples, which are intended to exemplify non-limiting embodiments of the invention.

### EXAMPLES

While the selective BRAF inhibitor vemurafenib is clinically active in metastatic BRAF V600 mutant melanoma patients, resistance is common. Multiple resistance mechanisms involve HSP90 clients and preclinical data show abrogation of resistance using concurrent treatment with the HSP90 inhibitor XL888. The combination of vemurafenib (960 mg bid) with escalating dose cohorts of XL888 (30 mg, 45 mg, 90 mg, 135 mg twice weekly) is being investigated in this ongoing study in unresectable stage III/IV BRAF V600 mutant melanoma patients.

The primary endpoint was safety and the determination of a maximum tolerated dose (MTD). Dose-limiting toxicity (DLT) was defined as a related grade 3 or greater adverse event in the first 8 weeks. Secondary endpoints included objective response (RECIST 1.1), survival, and pharmacodynamic biomarkers.

Fifteen patients have been enrolled (cohorts 1-3, n=3 each; cohort 4, n=6); median age of 60 years, 73% male, and primarily stage IV (IIIC(1), M1 A(2), M1B(2), and M1C(10)). In Cohort 1, vemurafenib was dosed at 960 mg twice daily and XL888 was dosed at 30 mg twice a week. In Cohort 2, vemurafenib was dosed at 960 mg twice daily and XL888 was dosed at 45 mg twice a week. In Cohort 3, vemurafenib was dosed at 960 mg twice daily and XL888 was dosed at 90 mg twice a week. In Cohort 4, vemurafenib was dosed at 960 mg twice daily and XL888 was dosed at 135 mg twice a week.

Most of the common adverse events were as expected for vemurafenib; for example, anorexia, fatigue, arthralgia, and rash. Diarrhea and vision changes were seen at all dose levels, with the highest rates being seen in cohort 4. These off-target effects resolved with the interruption of therapy. DLTs only occurred in cohort 4 (grade 3 diarrhea and pancreatitis), and an MTD has not yet been established. Fewer secondary cutaneous neoplasms were seen in higher dose cohorts. Objective response was observed in 11/13 evaluable patients (85%; 95% CI: 55-98%), including 2 CRs. In addition, one stage IIIC patient with a PR underwent resection of residual disease and pathology showed no viable tumor cells. Estimated 6- and 12-month PFS was 63% (95% CI: 28-84%) and 39% (95% CI: 11-68%), respectively.

Overall, early promising clinical activity with a manageable toxicity profile was observed. Additional patients will be enrolled to better define the MTD and evaluation of biomarker endpoints

The foregoing disclosure has been described in some detail by way of illustration and example for purposes of clarity and understanding. The invention has been described with reference to various specific and preferred embodiments and techniques. However, it should be understood that many variations and modifications can be made while remaining within the spirit and scope of the invention. It will be obvious to one of skill in the art that changes and modifications can be practiced within the scope of the appended claims. Therefore, it is to be understood that the above description is intended to be illustrative and not restrictive. The scope of the invention should, therefore, be determined not with reference to the above description, but should instead be determined with reference to the following appended claims, along with the full scope of equivalents to which such claims are entitled.

1. A method of treating BRAF V600 mutant melanoma comprising administering to a patient in need of such treatment a combination comprising vemurafenib in combination with the heat shock protein90 (HSP90) inhibitor XL888 or a pharmaceutically acceptable salt thereof, wherein vemurafenib is dosed orally at up to 960 mg twice daily and XL888 is dosed orally at up 90 mg twice a week.

2. The method of claim 1, wherein vemurafenib is dosed orally at 480 mg twice daily.

3. The method of claim 1, wherein vemurafenib is dosed orally at 720 mg twice daily.

4. The method of claims 1-3, wherein XL888 is dosed orally at 120 mg twice weekly.

5. The method of claims 1-3, wherein XL888 is dosed orally at 105 mg twice weekly.

6. The method of claims 1-3, wherein XL888 is dosed orally at 90 mg twice weekly.

7. The method of claims 1-3, wherein XL888 is dosed orally at 75 mg twice weekly.

8. The method of claims 1-3, wherein XL888 is dosed orally at 60 mg twice weekly.

9. The method of claims 1-3, wherein XL888 is dosed orally at 45 mg twice weekly.

10. The method of claims 1-3, wherein XL888 is dosed orally at 30 mg twice weekly.

11. The method of claims 1-10, wherein vemurafenib and XL888 are each present in a separate pharmaceutically acceptable composition and the compositions are administered simultaneously.

12. The method of claims 1-10, wherein vemurafenib and XL888 are each present in a separate pharmaceutically acceptable composition and the compositions are administered sequentially.

13. A method of treating melanoma in a human patient comprising administering an effective amount of a combination of vemurafenib and XL888 or a pharmaceutically acceptable salt thereof to the patient, wherein the combination of vemurafenib and XL888 has greater anticancer activity against said melanoma in the human patient than the maximum tolerated dose of vemurafenib against said melanoma in the human patient or the maximum tolerated dose of XL888 alone against said melanoma in the human patient.

14. The method of claim 1, wherein the combination of vemurafenib and XL888 has greater anticancer activity against said melanoma in the human patient than the optimum dose of each drug alone against said melanoma in the human patient.
